(19) United States
(12) Patent Application Publication
(10) Pub. No.: US 2022/0378018 A1
(43) Pub. Date: Dec. 1, 2022

(54) RAWHIDE ANIMAL CHEW INCLUDING MICROPORES FILLED WITH CHITOSAN

(71) Applicant: IMS TRADING, LLC, Jamesburg, NJ (US)

(72) Inventors: Glen S. AXELROD, Colts Neck, NJ (US); Ajay GAJRIA, Monmouth Junction, NJ (US)

(21) Appl. No.: 17/664,054

(22) Filed: May 19, 2022

Related U.S. Application Data
(60) Provisional application No. 63/201,970, filed on May 20, 2021.

(51) Int. Cl. A01K 15/02; A23K 50/42; A23K 40/00; A23K 20/163; A61K 8/73; A61Q 11/00

(52) U.S. Cl. CPC A01K 15/026; A23K 50/42; A23K 40/00; A23K 20/163; A61K 8/736; A61Q 11/00

(57) ABSTRACT

A microporous animal chew comprising a rawhide sheet having a thickness in the range of 0.1 millimeters to 3.0 millimeters and a plurality of micropores extending through said rawhide sheet at least partially filled with chitosan. The rawhide sheet provides a digestible yet chewable and durable animal chew.

### FIELD

The present disclosure is directed to a rawhide animal chew including micropores filled with chitosan.

### BACKGROUND

Animal chews are commonly provided to domesticated pets, and particularly dogs, to satisfy an innate need to chew. Animal chews also provide a source of entertainment and, depending on the material which the animal chew is formed from, can provide a source of nutrients. Further, it is believed that chewing on objects, such as animal chews, provides sufficient abrasion to improve the dental health and hygiene of an animal. Rawhide pet chews are sometimes perceived as a more natural product compared to pet chews formed from thermoplastic materials. Depending on preparation, rawhide also tends to be rather durable. Due to its durability, dogs may chew on rawhide for extended time periods slowly wearing away the rawhide. However, while dogs may gnaw on rawhide for extended periods of time, there are times relatively large pieces are pull off by the dog and ingested. It is, therefore, also beneficial to provide readily digestible rawhide chews without sacrificing durability.

Various means to solve the problem of improving digestibility, while maintaining chew-ability and durability, has been considered in the art. For example, U.S. Pat. No. 8,613,261 to Mendal, et al., described treating rawhide with various enzymes to break the rawhide down to increase its digestibility. U.S. Pat. No. 7,691,426 to Axelrod, et al., described comminuting rawhide into small particles or powder and incorporating it into edible resin. U.S. Pat. No. 7,678,402 to Marino describes forming large perforations in the chew to provide small sections that break off when chewed. Further, U.S. Pat. No. 7,147,888 to Brown, et al., describes impregnating rawhide pet chews with biofilm disrupting emulsions. To assist in impregnation of the rawhide, the rawhide is perforated with slits and holes. Brown, et al., also describes that increasing the surface area to volume ratio, increases the area contacted by the digestive juices of the animal, with the goal of improving digestion. Nevertheless, the problem of providing a digestible yet chewable and durable animal chew remains.

### SUMMARY

An aspect of the present disclosure relates to a microporous animal chew. The chew includes a rawhide sheet having a thickness in the range of 0.1 millimeters to 3.0 millimeters and a plurality of micropores extending through the rawhide sheet. The micropores exhibit a longest linear dimension across a cross-section of the micropores in the range of 1 micrometer to 2,000 micrometers and are present at a density of 1 to 100 pores per square centimeter. The micropores are at least partially filled with chitosan.

Another aspect of the present disclosure relates to a method of forming a microporous animal chew. The method includes providing a wet rawhide sheet having a thickness in the range of 0.1 millimeters to 4.0 millimeters including water present 60% to 80% by weight of the total weight of the rawhide sheet or greater. The rawhide sheet is then pierced with pins and micropores are formed in the rawhide sheet. The micropores have a largest linear cross-sectional length in the range of 1 micrometer to 2,000 micrometers and are arranged to provide a pore density in the range of 1 to 100 pores per square centimeter. The rawhide sheet is dried to include 1 to 20% by weight water of the total weight of the rawhide sheet. The micropores are then at least partially filled with chitosan.

### BRIEF DESCRIPTION OF THE DRAWINGS

The above-mentioned and other features of this disclosure, and the manner of attaining them, will become more apparent and better understood by reference to the following description of embodiments described herein taken in conjunction with the accompanying drawings, wherein:

FIG. 1 illustrates a method of forming an animal chew including a plurality of micropores;

FIG. 2A illustrates an embodiment of a cross-section of a micropore forming pin;

FIG. 2B illustrates an embodiment of a pore formed by the micropore forming pin exhibiting a longest linear cross-sectional length, CL;

FIG. 3A illustrates an embodiment of a press plate arrangement for forming micropores in a rawhide sheet;

FIG. 3B illustrates an embodiment of a press plate arrangement for forming micropores in a rawhide sheet;

FIG. 4 illustrates an embodiment of a calender roll system for forming micropores in a rawhide sheet; and

FIG. 5 illustrates pores formed in a rawhide sheet at an angle to the sheet surface.

### DETAILED DESCRIPTION

The present disclosure is directed to the provision of a rawhide animal chew including micropores at least partially filled with chitosan and a method of forming such an animal chew. The micropores increase the surface area to volume ratio of the rawhide, allowing for a larger surface area to be contacted with digestive juices of the animal, increasing enzymatic digestion of the animal chew. The micropores may be formed by a number of methods, including piercing rawhide sheets with pins while the rawhide is wet.

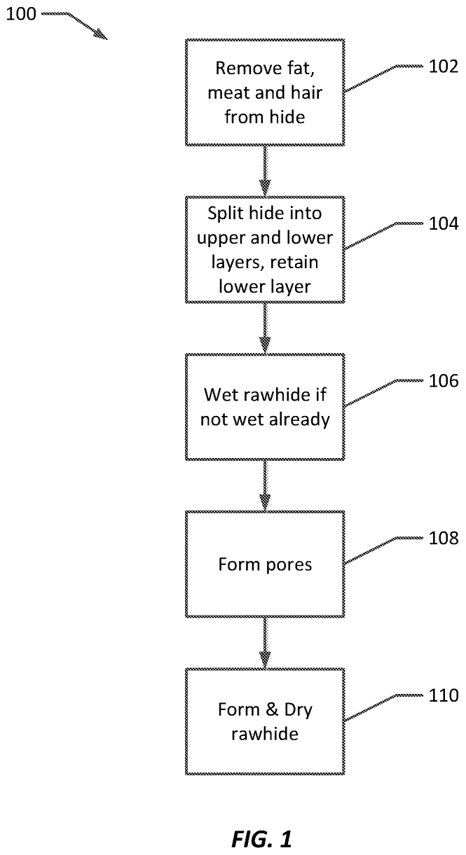

Rawhide, as referred to herein, is the untanned skin of buffalo, deer, elk, moose, cattle, pig, sheep, goats, or other hoofed animals. Generally, hides initially contain between 60% to 80% by weight water, such as between 60 to 70% by weight water, and 20% to 40% by weight other substances such as fibrous proteins, collagen, keratin, elastin and reticulin as well as 0.01 to 2% by weight ash including phosphorous, potassium, sodium arsenic, magnesium and calcium. The sheet containing such water has a thickness in the range of 0.1 mm to 4.0 mm. A hide may be prepared by any method known to those of ordinary skill in the art. One such method 100 is illustrated in FIG. 1 and includes removing most of the visible fat, meat and hair from the hide 102. The fat and meat may be removed by scraping and, once the fat and meat are removed, the hide may be treated in a solution of calcium carbonate, calcium hydroxide, slaked lime and wood ash, which may loosen and aid in the removal of hair. In addition, sodium sulphide, ammonium salts or enzymes may be added to the solution. However, in some preferred embodiments, enzymes and enzymatic treatments may be excluded. The hair is removed from the hide and the hide is rinsed. The hide may then be soaked in an aqueous solution including organic acids, inorganic acids and/or acid salts, such as potassium hydrogen tartrate and sodium bicarbonate. In embodiments, the hide is split 104 where the upper layer is separated from the under layer and the underlayer is used to make chews. In addition, the pieces may be soaked in a solution including hydrogen peroxide and chlorine. The hide may be rinsed again. The pieces of rawhide may be dried or further processed before drying. After drying, the hide preferably exhibits a moisture content in the range of 1% to 20% by weight water, including all values and ranges therein and preferably in the range of 5% to 18% by weight water. At such lower water levels the sheet has a preferred thickness of 0.1 mm to 3.0 mm.

Alternatively, the rawhide is provided in a rawhide resin composition, wherein the rawhide may be chopped or ground into small particles or powder. The particle size may be less than about 10 mm, such as in the range of 0.001 to 10 mm, including all values and increments therein. The moisture content of the rawhide may be adjusted to approximately 1% to 20% by weight of the rawhide, including all increments and values therein, such at 8%, 10%, etc. The rawhide may then be combined with up to 20% by weight of casein, such as in the range of about 0.1 to 20% by weight, including all values and increments therein. Casein may be understood as a phosphoprotein of milk, wherein a phosphoprotein may be described as a group of substances that are chemically bonded to a substance containing phosphoric acid. The rawhide may also be combined with gelatin up to 10% by weight, such as in the range of 0.1 to 10% by weight, including all values and increments therein. Gelatin may be understood as a protein product produced by partial hydrolysis of collagen. In addition, attractants, such as flavorants, or nutrients may be compounded with the rawhide.

The rawhide particles, casein, gelatin and any additional attractants or nutrients may be melt processed, wherein the particles are plasticated in a plasticating device. Suitable plasticating devices may include injection molding machines, extruders (twin-screw, single screw, etc.) or any other device which may provide sufficient thermal-mechanical interaction to cause plastication, such as blenders. The temperature of the plasticating device may be sufficient to melt at least 10% to 100% of the particles, including all values and increments therein and may be in the range of about 120 to 150° C., including all values and increments therein. In addition, the particles may be pressurized during plastication wherein the applied pressure may be in the range of about 1 to 20 MPa, including all values and increments therein.

The rawhide (the rawhide pieces or rawhide resin composition) is preferably provided in the form of sheets. Prior to drying, and while wet (e.g. at a water level of greater than or equal to 60%), the rawhide sheets preferably exhibit a thickness in the range of 0.5 mm to 4 mm, including all values and ranges therein. The dried sheets (e.g. at a water content of 1% to 20% by weight) therefore have a relatively lower thickness, in the range of 0.1 millimeters to 3.0 millimeters, including all values and ranges therein, and preferably in the range of 1.0 mm to 2.0 mm or even more preferably 0.1 to 0.5 mm. Referring back to FIG. 1, the pores are preferably formed in the rawhide while it is wet.

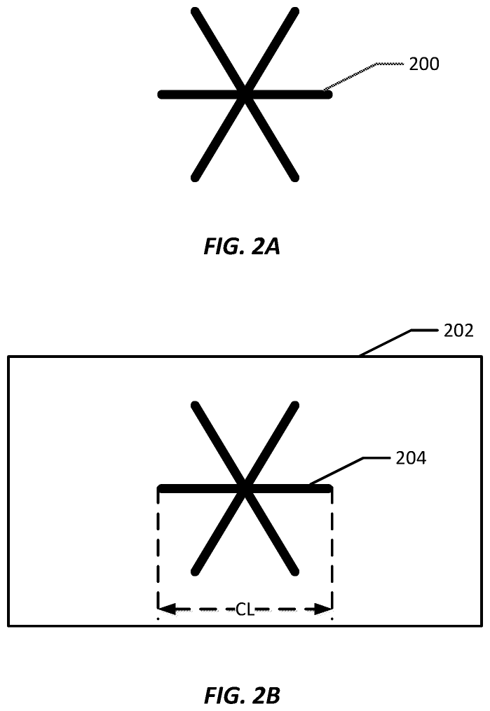

The rawhide is then pierced to form the micropores 108. Piercing may be facilitated by forcing a plurality of pins through the rawhide using, for example, a press or calendaring rolls. The micro-sized pins provide micropores having a largest linear cross-sectional length, CL, (see FIG. 2b) in the range of 1 micrometer to 2000 micrometers, including all values and ranges therein, such as from 1 micrometer to 300 micrometers, 100 micrometers to 300 micrometers, 300 micrometers to 1000 micrometers, and preferably in the range of 1000 micrometers to 2000 micrometers, once the rawhide shrinks upon drying. The pins may, therefore, be in the range of 0.1% to 50% larger in dimension that the desired pore size, including all values and ranges therein. The pins may exhibit a number of cross-sectional geometries.

Preferably, in embodiments, the pins and pores produced by them exhibit multiple arms to form an asterisk- or star-like geometry, including three or more arms and preferably from four to six arms. In other embodiments, the pins are preferably circular in cross-section but may alternatively be rectangular, triangular, ellipsoid quatrefoil or square in cross-section, or may be a combination of one or more of any of the shapes noted above. In any of the above embodiments, the pins may be hollow. A cross-section of an example of a pin 200 exhibiting six arms and the pore 202 produced by such a pin are illustrated in FIGS. 2a and 2b. The micropore is illustrated in FIG. 2b as exhibiting a longest linear cross-sectional length, CL, which in the case of a circular pore is the pore diameter.

The micropores may be spaced uniformly, or randomly, over the surface of the rawhide at a density in the range of 1 to 100 pores per square centimeter (cm²), including all values in the range of 20 to 50 pores per square centimeter and more preferably in the range of 2 to 20 pores per square centimeter. In embodiments, pore density remains constant over the entire sheet. Alternatively, pore density is varied across the surface of a sheet such that the density increases or decreases across the sheet surface.

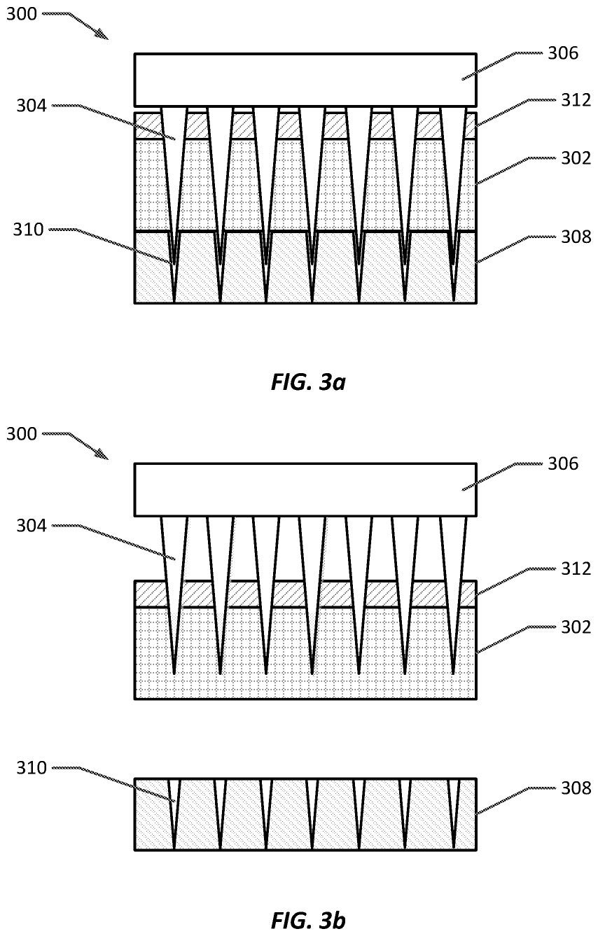

Generally, the pins are forced through the rawhide by utilizing equipment that may provide sufficient pressure to pierce the rawhide with the pins. As noted above, examples of such equipment include presses or calendaring rolls that may be operated using hydraulics, mechanical linkages or pneumatics. FIG. 3 illustrates an embodiment that utilizes a press 300. The press includes two plates 306 and 308 between which the rawhide 302 is placed. The plates are forced together to provide an elevated pressure of greater than 2 kPa, such as in the range of 2 kPa to 100 kPa, including all values and ranges therein, on the rawhide and pins. One plate, the carrier plate 306, carries the pins 304, and the other plate, the receiving plate 308, is the plate upon which the pins bear or are received. As illustrated in FIG. 3, the receiving plate may include a plurality of holes 310 for receiving the pins therein. In addition to applying force to pierce the rawhide, when the pins are received in the receiving plate 308, the plates may close together and squeeze excess water from the hide reducing the amount of water in the hide. The holes 310 may provide drain passages to facilitate water drainage out of the press. In alternative embodiments, both plates include both pins and holes. Further, in any of the above embodiments, the pins may retract into the carrier plate to facilitate stripping of the hide from the pins and the plate, or a stripper plate 312, as illustrated in FIG. 3, may be provided to help remove the rawhide from the pins once the press opens.

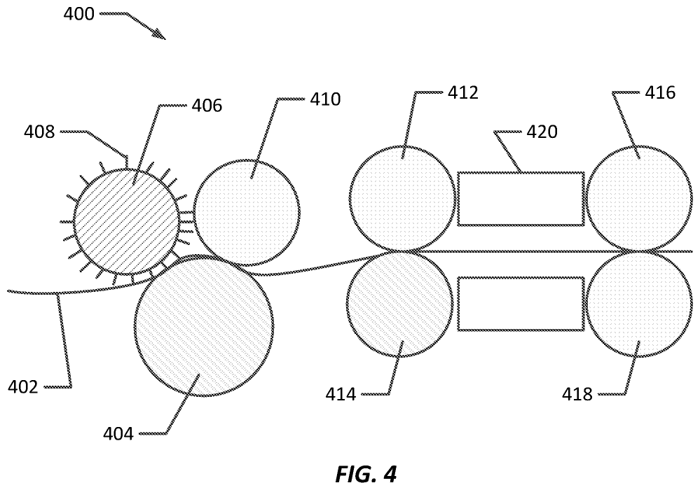

FIG. 4 illustrates an embodiment of calendaring equipment 400, wherein the rawhide 402 passes through a series of rolls. The rolls include at least one bearing roll 404 and at least one pin roll 406 on which a plurality of pins 408 are mounted. In addition, a squeeze roll 410 may optionally be provided. The pins 408, being carried by the pin roll 406, bear against the bearing roll 404 and pierce the rawhide 402 as the rawhide passes between the rolls. Then the squeeze roll 410, which is preloaded against the bearing roll 404 or an additional bearing roll (not illustrated), may be used to remove excess water from the rawhide. In embodiments, the pins may be retracted to facilitate release from the pin roll 406.

In embodiments, upon or after forming the micropores in the rawhide, the pores are at least partially filled or completely filled with chitosan. In addition, the surface between the pores may be coated with chitosan. Reference to completely filled micropores is reference to the feature that the micropores are filled with a sufficient amount of chitosan so that the filling is even or flush to the rawhide surface. Chitosan is reference to a polysaccharide composed of randomly distributed β-(1→4)-linked D-glucosamine (deacetylated unit). It is preferably sourced by deacetylation of chitin to about 50% of the free amine form. It may therefore have a heterogeneous type chemical structure made of both 1-4 linked 2-acetamido-2-deoxy-β-D-glucopyranose as well as 2-amino-2-deoxy-β-D-glucopyranose. Such heterogenous type chemical structure is illustrated below:

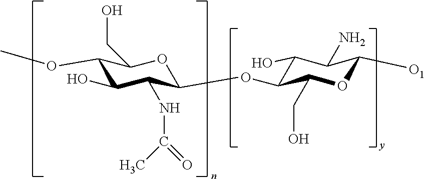

The chitosan offers a number of benefits. First, the chitosan itself is contemplated to provide antimicrobial activity, thereby serving to better preserve the rawhide after drying from microbial attack. In addition, as chewed upon by the animal, the chewed portions of the rawhide, which are contemplated then to have a greater susceptibility to microbial attack, are better preserved and the chewed portions are contemplated to be less likely to then mold and discolor. Second, as a polymeric material, the chitosan also can serve to prevent the pores, during drying of the rawhide, from closing. That is, the chitosan can also prevent the pores from “healing” themselves during drying so that the mircropores remain relatively intact. Accordingly, when ingested by the animal, the pores can still provide an increased surface area for exposure to digestive enzymes.

The chitosan may preferably be introduced into the pores and or onto the surface of the rawhide in a number of ways. Chitosan, as a solid, may be preferably dissolved into water solution and then sprayed onto the rawhide pores. The chitosan water solution may preferably contain 0.1% by weight to 5.0% by weight chitosan. Such water solution may preferably be formed by mixing chitosan in water along with relatively small amounts of an acid, such as acetic acid. Such water solution of chitosan may therefore be delivered to the surface of the rawhide containing the micropores, by, e.g., spraying, and upon evaporation, can provide a film of chitosan within the pores. In addition, one may form a film layer of chitosan along all or a portion of the surface of the rawhide. The chitosan film thickness, whether within the micropore, or on the surface of the rawhide, is contemplated to have a thickness in the range of 1 micrometer to 2000 micrometers, or 1 micrometer to 1000 micrometers, or 1 micrometer to 500 micrometers, or 1 micrometer to 250 micrometers, or 1 micrometer to 100 micrometers.

As noted, preferably, the micropores are at least partially filled with chitosan and/or completely filled with chitosan, depending upon the particular final level of chitosan that one may wish to achieve for the rawhide sheet that is being treated.

As the pet chews upon the rawhide, the chitosan is loosened from the animal chew and is readily ingested by the animal. The chitosan may also be preferably loaded into the micropores through pins that are hollow and injected upon pore formation or pressed into the pores through further calendering of the rawhide sheets.

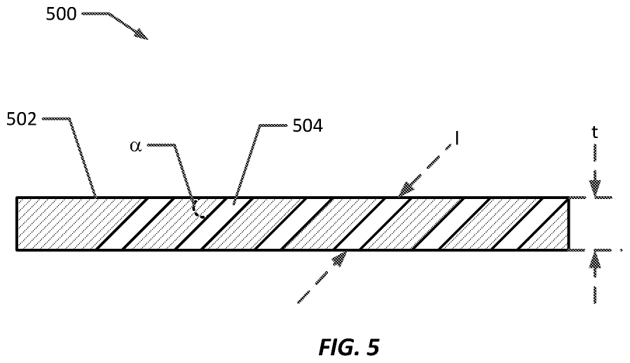

In addition, in any of the embodiments described above, the pins are preferably inserted into the rawhide at angle a relative to the surface 502 of the rawhide 500, thereby creating micropores having a length, l, greater than the average thickness, t, of the rawhide as illustrated in FIG. 5. The angle α, relative to the surface of the rawhide, may be in the range of 10 to 80 degrees, including all values and ranges therein such as 30 to 60 degrees. Providing the pores at an angle may assist in increasing the surface area to volume ratio as well as the surface area available for contact with digestive juices. The angle α is constant or, alternatively, varied over the surface of the sheet. As illustrated, the surfaces of the rawhide may generally be parallel. However, it should be appreciated that the thickness of the rawhide sheets may vary.

Again referring to FIG. 1, the rawhide sheets may be formed into a desired final shape and dried 110 after the pores are formed. The sheets may be dried from 3 to 6 days at temperatures in the range of 35° C. to 80° C., including all values and ranges therein. In embodiments, drying may occur under tension using a tension frame after removing the rawhide from a press or calendaring line. In other embodiments, as illustrated in FIG. 4, tenter rollers 412, 414, 416, 418 are placed in the process line after calendaring as illustrated in FIG. 4. To maintain a desired tension, the tentering rolls may rotate at faster speeds than the rolls which precede them. If other geometries are desired, the sheets may be cut, rolled and formed into rolls, rings, pretzels, sticks, braids, or chips. The rawhide may also be knotted to assume the general geometry of a bone or knotted bone. In alternative embodiments, the rawhide sheet may be die cut into desired shapes.

Once formed, the rawhide may then be dried with or without the assistance of a heat source such as an oven within the drying temperatures and times noted above. FIG. 4 illustrates the incorporation of an oven 420 in the process line, to facilitate drying of a sheet. The amount of water may be reduced to 1 to 20% by weight of the final product, including all values and ranges therein.

The above, therefore, provides a rawhide animal chew formed from a rawhide sheet having a thickness in the range of 0.1 millimeters to 3.0 millimeters. The rawhide sheet includes a plurality of micropores having a longest linear dimension across the cross-section length, CL, in the range of 1 micrometer to 2000 micrometers and present at a density in the range of 1 pore to 100 pores per square centimeter. The pores assume one or more geometries, depending on the pin utilized to form the pores. In embodiments, the rawhide is a rawhide resin composition. The micropores, in embodiments, are at least partially and/or completely filled with chitosan and the surface of the rawhide is partially and/or fully coated with chitosan. The chitosan, which is preferably in film form within the pores and/or on the sheet of the rawhide, is mechanically retained in the pores due to shrinking of the rawhide as it dries. The rawhide sheet may be manipulated to assume the geometry of a roll, ring, pretzel, or knotted bone or die cut to provide a desired geometry.

The micropores in the rawhide sheet may extend completely through and/or only partially through the rawhide sheet. Accordingly, the micropores may be open on two sides of the rawhide sheet of open only on one side. As alluded to above, the pores may have a longest linear dimension across a cross-section of the micropores in the range of 1 micrometer to 2,000 micrometers. In the case of a round shaped micropores, such may correspond to the diameter of the pores. The pores may also have a thickness or penetration depth into the rawhide of 0.1 millimeters to 3.0 millimeters, or 0.1 millimeters to 2.0 millimeters, or 0.1 millimeters to 1.0 millimeters, or 0.1 millimeters to 0.5 millimeters. By way of example, for a rawhide sheet having a thickness of 0.1 millimeters to 3.0 millimeters, the thickness or penetration depth of the micropores may be equal to such value (0.1 millimeters to 3.0 millimeters), in which case the micropores would be open on both sides of the sheet. Alternatively, the micropores may have a penetration depth that is less than the full thickness of the rawhide sheet and therefore be open on only one side of the rawhide sheet.

An evaluation has been made using an in vitro procedure with simulated gastric and small intestine digestive fluids. Reference is made to the in vitro testing procedures reported by Boisen and Eggum, 1991, Nutr. Res. Rev. 4 141-162. Samples were incubated for 6 hours in simulated gastric fluid containing hydrochloric acid and pepsin, then for 18 hours in simulated small intestinal fluid containing pancreatin. Following incubation, percentage in vitro dry matter disappearance was calculated. Dimensions and weights of each treat were measured before and after incubation. Table 1 provides the results for samples of rawhide having different pore sizes and different spacing between the pores in the identified samples:

### Table I

### Small Intestine Dry Matter Disappearance

TABLE I
Small Intestine Dry Matter Disappearance

| | Intestinal Phase (18 Hours) |
|---|---|
| | % Dry Matter |
| Sample | Disappearance |
| I (Control-No Pores) | 70.2 |
| II (2.0 mm pores/ | 84.3 |
| 6.0 mm apart) | |
| III (1.0 mm pores/ | 92.1 |
| 3.0 mm apart) | |

The microporous animal chews herein is one that is capable of indicating an intestinal phase dry matter disappearance in small intestinal fluid containing pancreatin, of 84.3% and as high as 92.1%, as compared to a control value of 70.2%. The average thickness of these tested samples was 1.7 mm. Accordingly, in preferred embodiment, it is contemplated that the microporous animal chews herein indicate a dry matter disappearance in simulated small intestinal fluid containing pancreatin in the range of greater than 70.2% to 95.0%, more preferably in the range of 75.0% to 95.0%, even more preferably 80.0% to 95.0%, and in a most preferred embodiment, in the range of 85.0% to 95.0% or 90.0% to 95.0%.

In particular, for 1.0 mm pores, where the pore size may range from 0.90 mm to 1.1 mm, that are spaced 3.0 mm apart (+/−0.5 mm) it was considered remarkable that one could achieve for such pore size, after 18 hours in the identified testing environment, a % dry matter disappearance of greater than 90% in small intestinal fluid containing pancreatin, and as noted, in the range of 90.0% to 95.0%. Moreover, it is contemplated here that when the pore size is reduced below 1.0 mm, and falls in the range of 0.001 mm to 0.99 mm, after 18 hours in the identified testing environment, the % dry matter disappearance will fall in the range of greater than 70.2% up to 100%. In addition, it is similarly contemplated that when one reduces the thickness below the average thickness of the samples tested (1.7 mm), to a thickness of 0.1 mm to less than 1.7 mm, the % dry matter disappearance will similarly fall in the range of greater than 70.2% up to 100%.

The foregoing description of several methods and embodiments has been presented for purposes of illustration. It is not intended to be exhaustive or to limit the claims to the precise steps and/or forms disclosed, and obviously many modifications and variations are possible in light of the above teaching. It is intended that the scope of the invention be defined by the claims appended hereto.

What is claimed is:

1. A microporous animal chew comprising:
a rawhide sheet having a thickness in the range of 0.1 millimeters to 3.0 millimeters;
a plurality of micropores in said rawhide sheet, wherein said micropores exhibit a longest linear dimension across a cross-section of said micropores in the range of 1 micrometer to 2,000 micrometers, are present at a density of 1 to 100 pores per square centimeter; and
wherein said micropores are at least partially filled with chitosan.

2. The microporous animal chew of claim 1 wherein said rawhide sheet has two sides and said micropores are present on one side of said rawhide sheet.

3. The microporous animal chew of claim 1 wherein said rawhide sheet has two sides and said micropores are present on both sides of said rawhide sheet.

4. The microporous animal chew of claim 1 wherein said chitosan is present as a film layer in said micropores.

5. The microporous animal chew of claim 1 wherein said plurality of micropores are completely filled with chitosan.

6. The microporous animal chew of claim 1 wherein said plurality of micropores extend partially through said rawhide sheet.

7. The microporous animal chew of claim 1 wherein said plurality of micropores extend completely through said rawhide sheet.

8. The microporous animal chew of claim 1 wherein said micropores have a penetration depth in said rawhide sheet of 0.1 millimeters to 3.0 millimeters.

9. The microporous animal chew of claim 1 wherein said rawhide sheet includes a surface region between said micropores and said surface region includes a layer of chitosan.

10. The microporous animal chew of claim 1, wherein said rawhide sheet is in the form of a roll, ring, pretzel, stick, braid, chip or knotted to assume the general geometry of a bone or a knotted bone.

11. The microporous chew of claim 1 wherein said chew has a water content of 1% to 20% by weight.

12. A method of forming a microporous animal chew, comprising:
providing a wet rawhide sheet having a thickness in the range of 0.5 millimeters to 4.0 millimeters including water present at 60% to 80% by weight of the total weight of the rawhide sheet or greater;
piercing said rawhide sheet with pins and forming micropores in said rawhide sheet, wherein said micropores have a largest linear cross-sectional length in the range of 1 micrometer to 2,000 micrometers and are arranged to provide a pore density in the range of 1 to 100 pores per square centimeter;
partially filling said micropores with chitosan; and
drying said rawhide sheet, wherein said rawhide sheet includes 1 to 20% by weight water of the total weight of the rawhide sheet.

13. The method of claim 12, further comprising removing a portion of water present in said rawhide sheet prior to drying.

14. The method of claim 12, further comprising stretching said rawhide sheet prior to drying.

15. The method of claim 10, wherein said rawhide sheet is provided between a carrier plate and a receiving plate, wherein said carrier plate carries said pins, and piercing said rawhide sheet includes forcing said plates together.

16. The method of claim 15, wherein said receiving plate includes holes to receive said pins therein.

17. The method of claim 15, further comprising compressing said rawhide sheet between said plates to remove at least a portion of water present in said rawhide sheet.

18. The method of claim 10, wherein said rawhide sheet is fed into calender rollers, wherein one of said rollers includes said pins and said pins pierce said rawhide sheet as said pins bear against a bearing roll.

19. The method of claim 18, further comprising compressing said rawhide sheet between a squeeze roll and said bearing roll and removing a portion of said water present in said rawhide sheet.

20. The method of claim 19, further comprising stretching said rawhide sheet prior to drying by passing said rawhide sheet between tenter rolls.

21. The method of claim 10, further comprising filling said micropores with a support additive prior to drying.

22. The method of claim 10, wherein said pins pierce said rawhide sheet at an angle α relative to a surface of said rawhide, wherein α is in the range of 10 to 80 degrees.

23. The method of claim 22, wherein said micropores exhibit a length that is greater than the thickness of said rawhide sheet.

24. The method of claim 12 wherein said wet rawhide sheet includes a rawhide resin composition.

25. The method of claim 10 wherein said dried rawhide has a thickness of 0.1 millimeters to 3.0 millimeters.
